(19) United States
(12) Patent Application Publication
(10) Pub. No.: US 2019/0021867 A1
(43) Pub. Date: Jan. 24, 2019

(54) Shoulder Joint Prosthesis

(71) Applicant: University Hospitals Coventry and Warwickshire NHS Trust, West Midlands (GB)

(72) Inventors: Tom Lawrence, West Midlands (GB); Richard King, Warwickshire (GB)

(21) Appl. No.: 16/071,556

(22) PCT Filed: Jan. 20, 2017
(86) PCT No.: PCT/GB2017/050139

§ 371 Date: Jul. 20, 2018

(30) Foreign Application Priority Data
Jan. 21, 2016 (GB) 1601159.5

(51) Int. Cl. A61F 2/40

(52) U.S. Cl. CPC A61F 2/4081; A61F 2002/3069; A61F 2/4059

(57) ABSTRACT

A reverse shoulder joint prosthesis (1) for location between a humerus and a scapula comprised a ball (5) to be fixed to the scapula and a humeral stem 6 to be fixed to the humerus. The prosthesis comprises a first cup (2), a second cup (3) and a plate (4) retained between said cups. The first cup is free to move about the ball. The plate has a trunion (16) adapted to connect to the humeral stem through an aperture in the second cup. The plate is free to move between the cups. The prosthesis has a first phase of movement in which the plate moves between the cups and a distinct second phase of movement in which the first cup moves on the ball. The shoulder joint prosthesis provides an extended range of movement through the provision of two motion phases while minimising wear, scapula infringement and instability.

### FIELD

The present invention relates to a shoulder joint prosthesis for use in reverse shoulder joint replacements, that is, replacements in which a prosthetic ball is attached to the scapula.

### BACKGROUND TO THE INVENTION

Reverse shoulder joint replacement is a well established method of replacing shoulder joints. 42% of all primary shoulder arthroplasty procedures in 2011 in the USA were reverse shoulder joint replacements. One of the reasons why reverse shoulder joint replacements are attractive is that they are more suitable for use in patients having significant rotator cuff damage.

However, patients receiving a reverse shoulder joint replacement often suffer from a restricted range of movement, as a consequence of the relatively small range of movement of the replacement glenohumeral articulation. Another common issue experienced following a reverse shoulder joint replacement is impingement of the humeral component on the scapula resulting in scapula notching. In severe cases scapula notching can lead to loosening of the glenoid component in the joint replacement. As with all shoulder joint replacements reverse shoulder joint replacements can lack stability.

Hence, there is a need for reverse shoulder joint replacement prostheses which offer the prospect of improvement in one or more of these respects.

### SUMMARY OF THE INVENTION

According to the present invention there is provided a shoulder joint prosthesis as set forth in the appended claims. Also disclosed is a method of using the shoulder joint prosthesis. Other features of the invention will be apparent from the dependent claims, and from the description which follows.

Throughout this specification, the term “comprising” or “comprises” means including the component(s) specified but not to the exclusion of the presence of other components. The term “consisting essentially of” or “consists essentially of” means including the components specified but excluding other components except for materials present as impurities, unavoidable materials present as a result of processes used to provide the components, and components added for a purpose other than achieving the technical effect of the invention.

The term “consisting of” or “consists of” means including the components specified but excluding other components.

Whenever appropriate, depending upon the context, the use of the term “comprises” or “comprising” may also be taken to include the meaning “consists essentially of” or “consisting essentially of”, and also may also be taken to include the meaning “consists of” or “consisting of”.

The optional features set out herein may be used either individually or in combination with each other where appropriate and particularly in the combinations as set out in the accompanying claims. The optional features for each aspect or exemplary embodiment of the invention, as set out herein are also applicable to all other aspects or exemplary embodiments of the invention, where appropriate. In other words, the skilled person reading this specification should consider the optional features for each aspect or exemplary embodiment of the invention as interchangeable and combinable between different aspects and exemplary embodiments.

It is one aim of the present invention, amongst others, to provide a shoulder joint prosthesis which at least partially obviates or mitigates at least some of the disadvantages of the prior art, whether identified herein or elsewhere. For instance, it is an aim of embodiments of the invention to provide a shoulder joint prosthesis which can extend the range of movement of a replaced shoulder joint. In particular it is an aim to extend the range of movement following a reverse shoulder joint replacement. It is a further aim of embodiments of the invention to provide a shoulder joint prosthesis with a low wear rate and reduced impingement on the scapula.

A first aspect of the invention provides a shoulder joint prosthesis for location between a humerus and a ball member in a scapula, the prosthesis comprising a first cup and a second cup and a plate retained between said cups;
wherein the first cup is adapted to move about the ball member;
wherein the plate is adapted to connect to the humerus through the second cup; and
wherein the plate is adapted to move about the first cup.

The first and second cups may each be generally dish- or bowl-shaped. Preferably the first and second cups each comprise a convex surface facing the humerus and a concave surface facing the ball member. The concave surface of the first cup enables its movement about the ball member. The plate is able to move between the convex surface of the first cup and the concave surface of the second cup.

The plate may comprise a convex and concave surface. Preferably the plate comprises a convex surface facing the humerus and a concave surface facing the ball member. The concave surface of the plate enables its movement about the convex surface of the first cup.

The plate may comprise a trunion located through the aperture of the second cup. The plate is adapted for attachment directly or indirectly to the humerus. The plate may comprise a trunion adapted for attachment directly or indirectly to the humerus. The trunion may be formed as an integral feature of the plate. Preferably, in use, the plate is secured to the humerus via a humeral stem adapted to be inserted into the humerus. The trunion may comprise a taper to facilitate securement to the stem. Preferably the trunion comprises a Morse taper. The trunion may taper in the direction away from the convex surface of the plate. The trunion is secured to the stem to limit instability of the prosthesis.

In embodiments of the invention the first cup may be in contact with not more than 40% of the contactable area of the ball member at any time. Preferably the first cup is in contact with not more than 30% of the contactable area of the ball member at any time.

In embodiments of the invention the plate may be in contact with not more than 40% of the convex surface of the first cup at any time. Preferably the plate is in contact with not more than 30% of the convex surface of the first cup at any time.

In embodiments of the invention the plate may be in contact with not more than 40% of the concave surface of the second cup at any time. Preferably the plate is in contact with not more than 30% of the concave surface of the second cup at any time.

Example embodiments aim to provide relatively low contact areas, which may assist in achieving a wide range of movement, in use. A further aim of the provision of relatively low contact areas is to reduce the rate of wear of the shoulder joint prosthesis and thus reduce potential instability of the prosthesis.

The plate may be retained between the first and second cups so that it is engaged with surfaces of both cups simultaneously. Preferably the first and second cups are adapted to attach to each other with the plate retained between them. The convex surface of the first cup may be complementary to the concave surface of the second cup so that they fit together securely with a space between them in which the plate is captive, but with freedom to move. The first and second cups may attach together around their respective peripheries. Suitably the first and second cups have inter-engagement means which extend around the respective peripheries or rims of the first and/or second cups. Preferably the first and second cups are secured together by a snap-fit connection or by an interference fit or by screw fitting. An interference fit may be augmented by an additional locking ring. Suitably the first and second cups are secured together by a snap-fit connection.

One of the cups may comprise a groove wherein the groove is adapted to be connected to an abutment on the other cup. The groove may define an area of reduced thickness at the periphery of a cup. The groove enables the cups to be securely attached together and for the plate to be retained between said cups. The groove may act as a socket for the abutment on a corresponding cup. Preferably the groove extends around the concave inner surface of the second cup. Suitably the groove extends around the entire circumference of the second cup.

As noted the second cup comprises an aperture through which the plate can be connected to the humerus. The plate may be connected to the humerus through the agency of the trunion of the plate.

One or both cups may comprise an abutment wherein in use contact of the plate with the abutment initiates movement of the first cup about the ball member. Preferably the abutment is provided at a periphery of the first cup. The abutment may extend around the entire circumference or rim of the first cup. The first cup may comprise an abutment as an integral feature of the first cup. Preferably the abutment is adapted so that in use the abutment is enclosed by the second cup. Suitably the abutment enables the second cup to be attached securely to the first cup. Suitably the abutment is comprised by said inter-engagement means which extend around the respective peripheries or rims of the first and/or second cups. Preferably the abutment comprises a ridge which enables the inter-engagement of the first cup with the second cup. The dimensions and shape of the ridge may correspond to the dimensions and shape of the groove on the other cup thus permitting the cups to be securely attached together.

Thus the movement of the plate about the first cup—which may be regarded as a first phase of movement—may be terminated by the engagement of the plate with the abutment at the periphery of the first cup. In some embodiments movement of the plate about the first cup may be terminated by simultaneous engagement of the plate (preferably the trunion thereof) with the aperture within the second cup. In preferred embodiments termination of the movement of the plate about the first cup is terminated only by the engagement of the plate with the abutment at the periphery of the first cup. Advantageously, in use, the plate can be confined to relatively small movements about the first cup.

Termination of the first phase of movement suitably also initiates a second phase of movement, in which the first cup moves about the ball member. Preferably the second phase of movement does not involve the movement of the plate about the first cup. Preferably it comprises only the movement of the first cup about the ball.

The prosthesis may include said ball member, which is adapted for securement to a scapula. The ball member may be adapted for securement to the shoulder socket or glenoid cavity of the scapula. Securement of the ball member to the glenoid cavity of the scapula provides a reverse shoulder joint configuration. The ball member may be an artificial ball member or glenosphere. The ball member may comprise a metallic material or a ceramic material or a polymeric material. Preferably the ball member may comprise a metallic material. The ball member may preferably comprise cobalt chrome.

The plate may comprise a metal. The plate may comprise stainless steel, tantalum, titanium, vanadium, cobalt, chromium, tungsten, nickel, and molybdenum. Preferably the plate comprises cobalt chrome.

The first cup may comprise a polymeric material, for example a polyolefin. Preferably the first cup comprises polyethylene. The first cup may comprise an ultra-high molecular weight polyethylene. The first cup may comprise vitamin E crosslinked polyethylene.

The second cup may comprise a polymeric material, for example a polyolefin. Preferably the second cup comprises polyethylene. The second cup may comprise an ultra-high molecular weight polyethylene. The second cup may comprise vitamin E crosslinked polyethylene.

The prosthesis according to the first aspect of the invention suitably provides first and second motion phases, wherein:
the first phase comprises the movement of the plate about the first cup and is limited by the extent of the movement of the plate on the first cup; and
the second phase comprises the movement of the first cup about the ball member and is initiated by the extent of the movement of the plate on the first cup.

An aim of example embodiments of the present invention is to provide a shoulder joint prosthesis with a greater range of movement than existing prostheses, without increasing wear of the prosthesis. This may be achieved because of the provision of two phases of movement—firstly the movement of the plate about the first cup enabling movements of the shoulder joint over a small range, and secondly movement of the first cup over the ball member, enabling coarser movements.

Thus in preferred embodiments the first motion phase suitably takes place by the movement of the plate about the first cup until the plate has reached its limit of movement with respect to the first cup. The second motion phase suitably takes place by the movement of the first cup about the ball member. The second phase of movement enables a larger extent of movement of the shoulder joint. Preferably the second phase of movement increases the range of movement compared with existing prostheses having only one phase of movement. The movement of the shoulder joint prosthesis is suitably not the result of movement of both the plate and first cup occurring simultaneously but instead is the result of the separate and sequential movement of the plate about the first cup, and of the first cup about the ball member.

Advantageously the profile of the shoulder joint prosthesis is reduced compared to current reverse shoulder joint prostheses thus potentially incurring less soft tissue impingement and irritation around the margin of the prosthesis.

A second aspect of the invention provides a shoulder joint prosthesis for placement between a humerus and a scapula, the prosthesis comprising:

a ball member securable to a scapula;
a humeral insert securable to a humerus; and
a first cup and a second cup and a plate retained between said cups;
wherein the first cup is free to move about the ball member;
wherein the plate is adapted to connect to the humeral insert through the second cup; and
wherein the plate is free to move about the first cup.

A third aspect of the invention provides a method for replacing a shoulder joint with a shoulder joint prosthesis according to the first aspect of the invention. Suitably the method may comprise:
locating a shoulder joint prosthesis between a humerus and a ball member in a scapula;
wherein the prosthesis comprises a first cup and a second cup and a plate retained between said cups;
wherein the first cup and second cup are attached together;
wherein the prosthesis is secured to the humerus by direct or indirect attachment of the plate to the humerus through the second cup;
wherein the first cup is free to move about the ball member; and
wherein the plate is free to move about the first cup.

Also disclosed herein is the use of a shoulder joint prosthesis of the first aspect in a reverse shoulder joint replacement procedure.

In preferred methods the ball member is part of the prosthesis and is secured to the scapula.

### BRIEF DESCRIPTION OF THE DRAWINGS

For a better understanding of the invention, and to show how exemplary embodiments of the same may be brought into effect, reference will be made, by way of example only, to the accompanying diagrammatic figures, in which:

FIG. 1 shows the components of a shoulder joint prosthesis according to an embodiment of the invention;

FIGS. 2a and 2b are views of a first cup according to the embodiment of FIG. 1;

FIGS. 3a and 3b are views from different angles of a second cup according to the embodiment of FIG. 1;

FIGS. 4a and 4b are views from different angles of a plate according to embodiment of FIG. 1;

FIG. 5 shows the first cup, the plate and the second cup according to the embodiment of FIG. 1 in a perspective expanded view;

FIG. 6 depicts the shoulder joint prosthesis according to the embodiment of FIG. 1 wherein the first cup and the second cup are attached together enclosing the plate therebetween; wherein the first cup is in contact with a ball member and showing a humeral stem to be engaged with the plate;

FIG. 7 depicts the assembled shoulder joint prosthesis according to the embodiment of FIG. 1 wherein the first cup and the second cup are attached together retaining the plate, the first cup is in contact with the ball member and wherein the humeral stem is engaged with a trunion of the plate; and

FIGS. 8 to 11 depict the shoulder joint prosthesis of FIG. 1 wherein the humeral stem is in different positions corresponding to movement of an attached humerus.

### DETAILED DESCRIPTION OF THE INVENTION

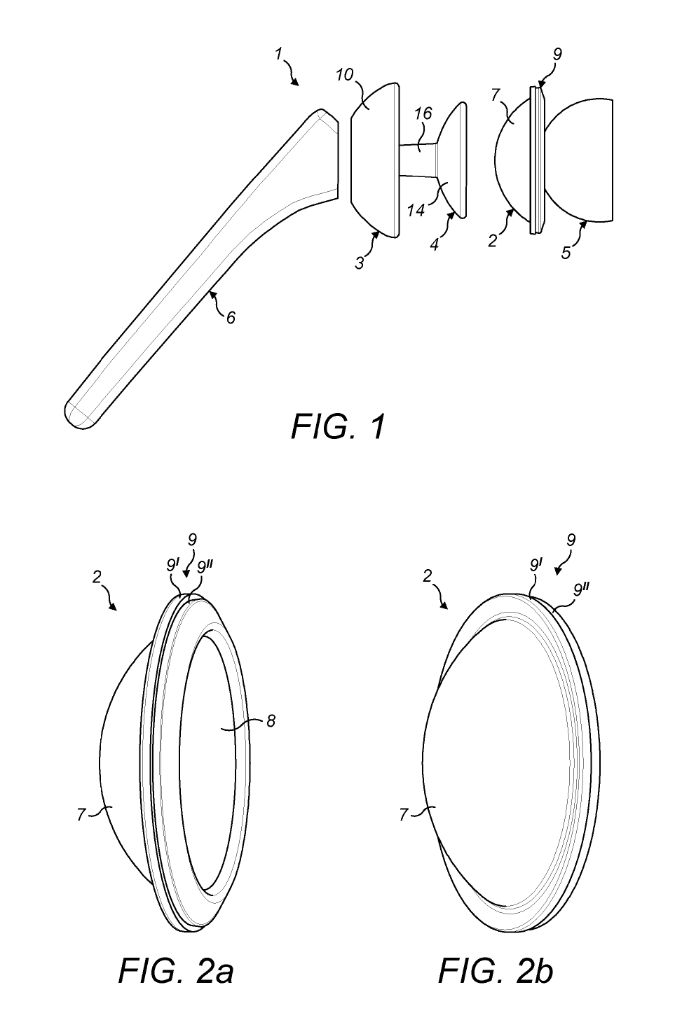

FIG. 1 shows in an expanded view the components of a prosthesis 1 comprising a first cup 2 and a second cup 3 and a plate 4 to be retained between the cups 2 and 3. The first cup 2, second cup 3 and plate 4 are located between a ball member 5 and a humeral stem 6.

The prosthesis is a reverse shoulder prosthesis. Thus the ball member 5 is secured in use to the scapula of a patient, and the stem 6 is introduced into the humerus.

FIGS. 2a and 2b show the first cup 2 comprising a convex surface 7 and a concave surface 8. In use the concave surface 8 is adapted to freely move about the ball member 5. The convex surface 7 is adapted so that the plate 4, held captive between the cups 2 and 3, can freely move about the convex surface 7. The first cup 2 comprises an abutment 9 extending outwards about the periphery of the first cup 2. The abutment 9 extends completely about the circumference of the first cup 2. The abutment 9 is formed as a continuous rim of the first cup 2. As shown the abutment 9 may comprise a ridge 9′ and a comparatively lower portion 9″. As will be further explained the abutment 9 facilitates attachment of the second cup 3 to the first cup 2.

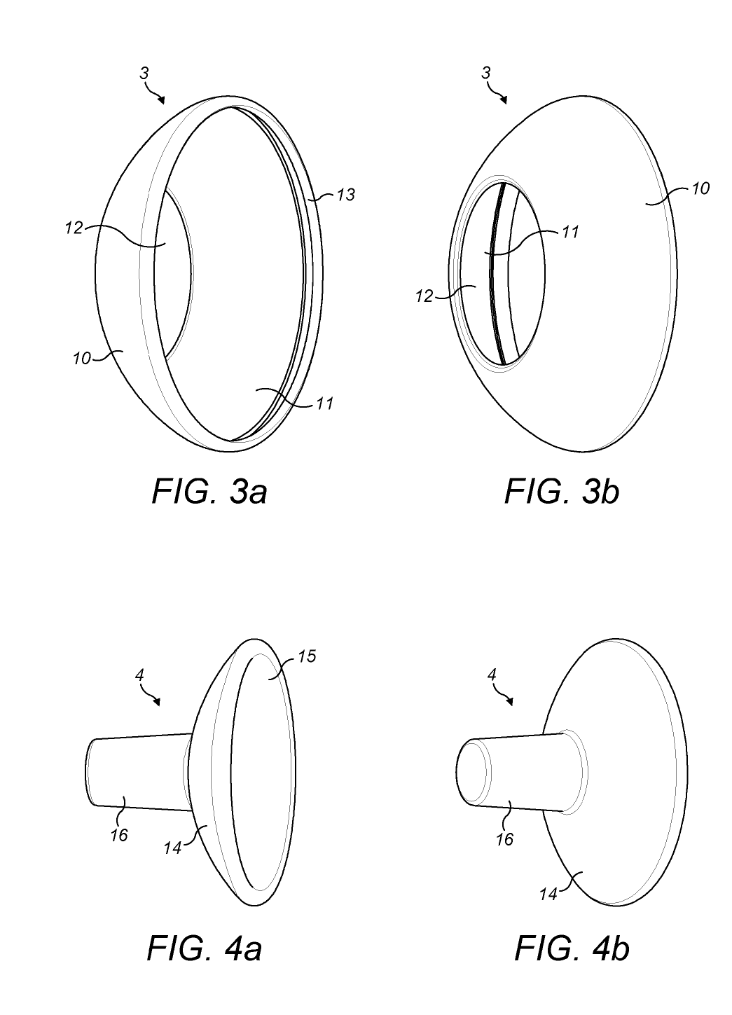

As shown in FIGS. 3a and 3b the second cup 3 comprises a convex surface 10 and a concave surface 11. The second cup 3 comprises an aperture 12. The second cup comprises a groove 13 extending around the edge of the concave surface 11. The groove 13 comprises an area of reduced thickness compared to the rest of the second cup 3. The groove 13 extends completely about the circumference of the second cup 3. The groove 13 corresponds to the abutment 9 of the first cup 2. In use the first cup 2 and second cup 3 are attached securely to each other by means of the complementary abutment 9 and groove 13. In use the plate 4 is retained by the attachment of the second cup 3 to the first cup 2. The plate is captive between the cups but is free to move between them, in engagement with their respective opposed convex and concave surfaces.

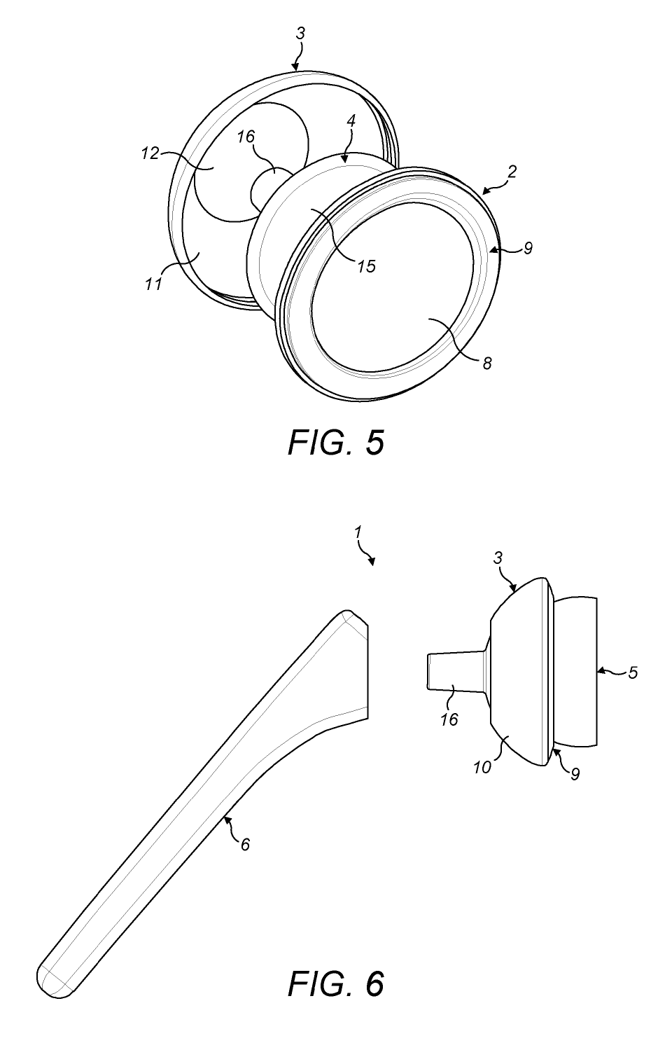

As shown in FIGS. 4a and 4b the plate 4 comprises a convex surface 14 and a concave surface 15. The plate 4 comprises a trunion 16 projecting from its convex surface 14. The trunion 16 has a Morse taper, away from the convex surface 14 of the plate. The trunion 16 is formed as an integral part of the plate 4.

FIG. 5 shows a perspective view of the first cup 2, plate 4 and second cup 3 looking generally towards the concave surface 8 of the first cup. The plate 4 is located between the first cup 2 and second cup 3. The trunion 16 of plate 4 extends through the aperture 12 of second cup 3.

FIG. 6 shows the configuration of the prosthesis 1 when the first cup 2 and second cup 3 have been attached together. The plate 4 is retained between the first cup 2 and second cup 3. The trunion 16 of plate 4 protrudes through the aperture 12 of second cup 3. The first cup 2 is almost completely enclosed within second cup 3. The second cup 3 attaches to the first cup 2 by means of the abutment 9 so that only a portion of the abutment 9 of the first cup 2 remains visible, when seen from the side. The first cup 2 is in contact with the ball member 5. The stem 6 is shown ready for insertion into the humerus and attachment to the trunion 16 of the plate 4.

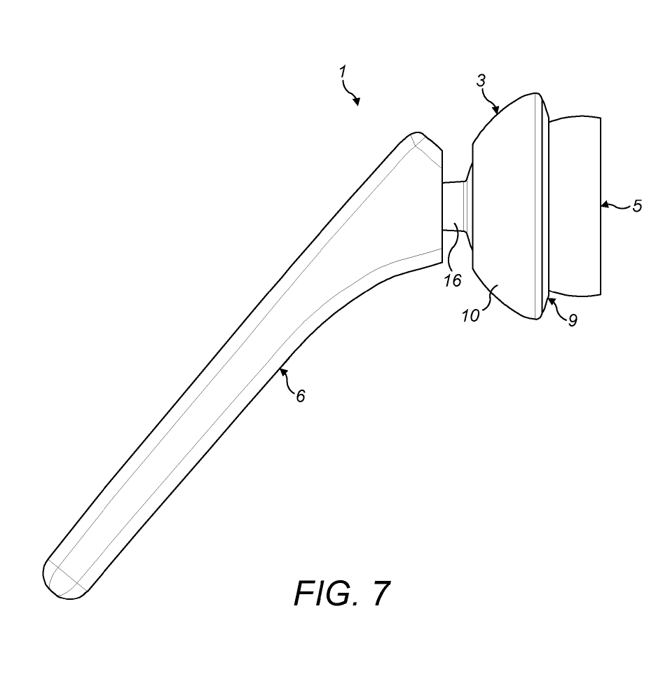

FIG. 7 shows the shoulder joint prosthesis 1 fully assembled and located between the stem 6 and ball member 5. The trunion 16 of plate 4 is attached to the stem 6. The stem 6 is adapted to be inserted in the humerus. The attachment of trunion 16 of plate 4 to the stem 6 secures the shoulder joint prosthesis 1 to the humerus.

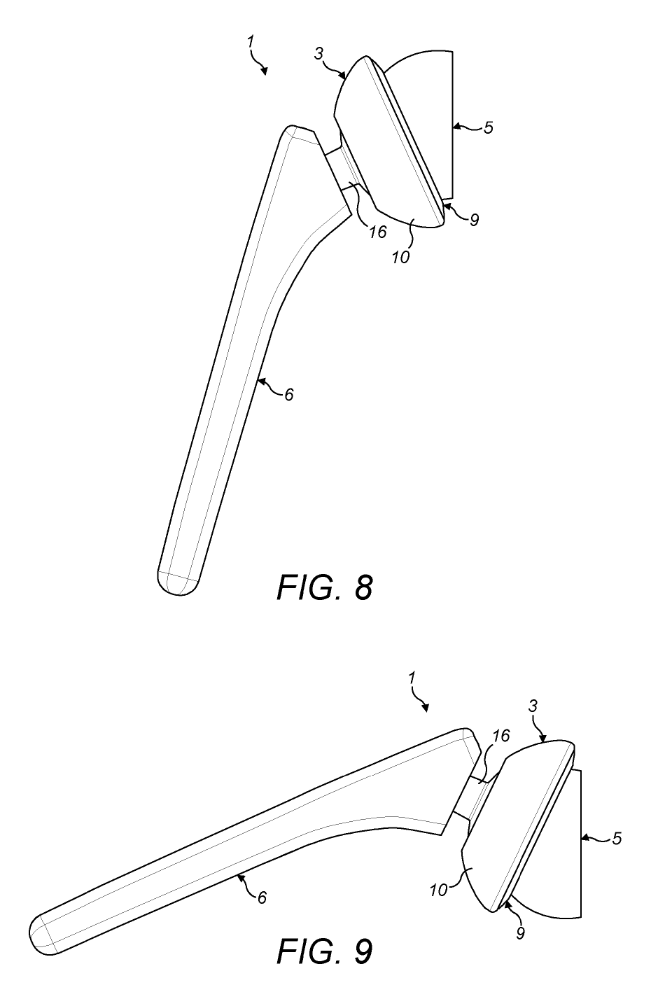

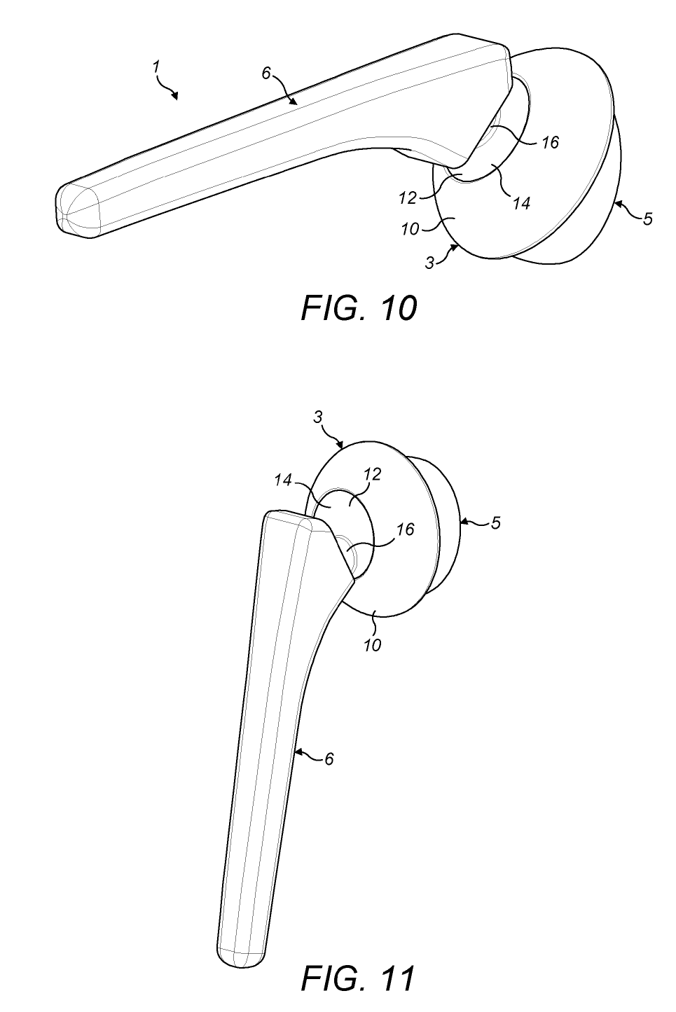

FIGS. 8 to 11 show movement of the stem 6 causing movement of the trunion 16 and consequently of the plate 4 causing the first cup 2 to move about the ball member 5. The plate 4 is free to move about the first cup 2 until the edge of the plate abuts the abutment 9 of the first cup 2. The plate-halting mechanism limits the movement of the plate 4 about the first cup 2. Small movements are thus enabled and wear problems are limited by stopping the free movement of the plate 4 about the first cup 2.

In use in a reverse shoulder replacement configuration the ball member 5 is secured to the scapula of a patient, and the stem 6 is introduced into the humerus.

In use, when a larger range of movement is required the plate 4 impacts against the abutment 9 of the first cup 2. This action is not visible in the figures as the plate 4 and the first cup 2 are located within the second cup 3. By these means the movement of the plate is stopped and a second phase of motion is initiated involving the free movement of the first cup 2 about the ball member 5. The second phase of motion enables a wider range of movement, limited by the rotator cuff of the shoulder. The plate 4 remains at the abutment 9 during this second phase of motion. The plate 4 will begin to move again when the stem 6 moves back to a central location corresponding to movement of the attached humerus.

Although a preferred embodiment has been shown and described, it will be appreciated by those skilled in the art that various changes and modifications might be made without departing from the scope of the invention, as defined in the appended claims.

In summary, the invention provides a shoulder joint prosthesis for use in a reverse shoulder joint replacement. The shoulder joint prosthesis provides movement over a wide range by providing a mechanism for two phases of motion but without increasing wear and with less scapula impingement and instability

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims and drawings), and/or all of the steps of any method or process so disclosed, may be combined in any combination, except combinations where at least some of such features and/or steps are mutually exclusive.

Each feature disclosed in this specification (including any accompanying claims, and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims and drawings), or to any novel one, or any novel combination, of the steps of any method or process so disclosed.

1. A shoulder joint prosthesis for location between a humerus and a ball member in a scapula,
the prosthesis comprising a first cup and a second cup and a plate retained between said cups;
wherein the first cup is adapted to move about the ball member;
wherein the plate is adapted to connect to the humerus through the second cup; and
wherein the plate is adapted to move about the first cup.

2. A shoulder joint prosthesis according to claim 1 wherein the first and second cups each comprise a convex surface facing the humerus and a concave surface facing the ball member.

3. A shoulder joint prosthesis according to claim 1 wherein the first cup is in contact with not more than 40% of the contactable area of the ball member at any time.

4. A shoulder joint prosthesis according to claim 1 wherein the first and second cups are adapted to attach to each other with the plate retained between them.

5. A shoulder joint prosthesis according to claim 4 wherein the first and second cups attach together around their respective peripheries.

6. A shoulder joint prosthesis according to claim 1 wherein one or both cups comprise an abutment wherein in use contact of the plate with the abutment initiates free movement of the first cup about the ball member.

7. A shoulder joint prosthesis according to claim 1 wherein the second cup comprises an aperture through which the plate can be connected to the humerus.

8. A shoulder joint prosthesis according to claim 1 wherein the plate comprises a trunion adapted for attachment directly or indirectly to the humerus.

9. A shoulder joint prosthesis according to claim 8 wherein the plate is secured to the humerus via a humeral stem adapted to be inserted into the humerus.

10. A shoulder joint prosthesis according to claim 1 wherein the prosthesis includes said ball member, which is adapted for securement to the scapula.

11. A shoulder joint prosthesis according to claim 1 wherein the plate comprises a metal.

12. A shoulder joint prosthesis according to claim 1 wherein the first and second cups each comprise a polymeric material.

13. A shoulder joint prosthesis according to claim 1 adapted for use in a reverse shoulder joint replacement.

14. A shoulder joint prosthesis according to claim 1 adapted to provide two phases of movement, wherein the first phase comprises the movement of the plate about the first cup and the second phase comprises the movement of the first cup about the ball member.

15. A shoulder joint prosthesis according to claim 6 wherein in use the motion of the prosthesis comprises first and second motion phases, wherein:
the first phase comprises the movement of the plate about the first cup and is limited by the extent of the movement of the plate on the first cup; and
the second phase comprises the movement of the first cup about the ball member and is initiated by the extent of the movement of the plate on the first cup.

16. A shoulder joint prosthesis for placement between a humerus and a scapula, the prosthesis comprising:
a ball member securable to a scapula;
a humeral insert securable to a humerus; and
a first cup and a second cup and a plate retained between said cups;
wherein the first cup is free to move about the ball member;
wherein the plate is adapted to connect to the humeral insert through the second cup; and
wherein the plate is free to move about the first cup.

17. A method for replacing a shoulder joint with a shoulder joint prosthesis comprising:
locating a shoulder joint prosthesis between a humerus and a ball member in a scapula;
wherein the prosthesis comprises a first cup and a second cup and a plate retained between said cups;
wherein the first cup and second cup are attached together;
wherein the prosthesis is secured to the humerus by direct or indirect attachment of the plate to the humerus through the second cup;
wherein the first cup is free to move about the ball member; and
wherein the plate is free to move about the first cup.

18. (canceled)
